(19) United States
(12) Patent Application Publication
(10) Pub. No.: US 2006/0031952 A1
(43) Pub. Date: Feb. 9, 2006

(54) Methods for producing transgenic animals

(76) Inventors: Pawel Pelczar, Riehen (CH); Stefan Moisyadi, Honolulu, HI (US); Barbara Hohn, Arlesheim (CH); Takehito Kaneko, Kumamoto (JP); Ryuzo Yanagimachi, Honolulu, HI (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/151,936

(22) Filed: Jun. 14, 2005

Related U.S. Application Data
(60) Provisional application No. 60/580,116, filed on Jun. 16, 2004.

(51) Int. Cl. A01K 67/027; C12N 5/08; C12N 5/06

(52) U.S. Cl. 800/14; 800/21; 435/364; 435/366

(57) ABSTRACT

The invention provides methods for producing transgenic embryos and animals with a higher efficiency than presently available procedures based on Intracytoplasmic Sperm Injection (ICSI) of nucleic acid complexed with recombinase.

### FIELD OF THE INVENTION

The current invention relates to integration of foreign DNA in animal embryos useful in the production of transgenic cells and animals and methods for using the transgenic animals as models for human disease and diagnosis.

### BACKGROUND OF THE INVENTION

There are several reported methods of for producing transgenic animals by introducing recombinant DNA into their somatic and germinal cells. One successful method makes use of Lentiviral-mediated transgenesis, where disarmed retroviral vectors are used to insert desirable genes into the host organism, usually at the single celled embryo stage. The most commonly used technique however, uses pronuclear microinjection entailing injection of the transgene DNA into one of the pronuclei of fertilized oocytes. Both methods for producing transgenic animals have their limitations.

For the Lentiviral technique, the relatively small amount of transgene DNA that can be transported due to the limited physical volume of the viral particles and the high lethality demonstrated by developing embryos is a serious drawback. To overcome the size limitation imposed on the transgenes, sequences of interest have been placed under the control of viral long terminal repeats (LTRs). Although increasing the amount of transgene DNA that can be shuttled by the viral vector, this approach has brought about expression problems that can be alleviated by the use of mutated LTRs. Such events however, necessitate extra steps in the preparation of transgene DNA. This coupled to the required specialized containment facilities for retroviral production makes it prohibitive for most laboratories to carry out retroviral transgenesis. There are also lingering concerns about the potential consequences of recombination events between the viral vector and endogenous retroviruses, leading to the generation of new and more potent pathogenic agents.

Transgenesis using pronuclear microinjection although widely used is of low efficiency attributable to the poor survival of embryos to term and low transgene integration rates. The current efficiency of producing transgenic mice with this technique is about 4% of oocytes injected. This low efficiency is due in part to the lack of control over injected DNA concentration, size of injected DNA, injection buffer used, the difficulty in some species of pronuclei visualization (e.g., cattle and pigs), the timing of the injection following sperm penetration and the damage caused to both transgene DNA and chromosomal DNA by the very fine tipped microinjection pipettes used. In addition, the efficiency is further affected by only 70% of the founders produced transmitting their transgene to offspring, of which only approximately 50% express their transgene at useful levels.

More recently, improved embryo survival and transgene integration was obtained using pronuclear microinjection after preincubating the denatured transgene with RecA (E. Maga, 2001, Cloning and Stem Cells 3:233-241; Maga et al., 2003, Transgenic Research 12:485-496). However, most founder animals were mosaic and transmitted the transgene to their offspring at low frequencies. Mosaicism was thought to occur because RecA increased the probability that the transgene integrates into the genome after the one-cell stage in this system, as well as the recombinase reaction being related to cell division after the one-cell stage. The level of mosaicism observed using this method is a severe limitation for the efficient production of stably transgenic animals.

Transgenic embryonic stem cells (ESC) obtained by transfection with DNA constructs have also been used to obtain chimeric animals. The method involves the injection of genetically engineered ESCs harboring a desired mutation into fertilized embryos at the morula stage (about 20 to 50 cells) or the blastocyst stage (about 100 cells) of embryonic development. Upon implantation, such embryos often give rise to chimeric animals whose subsequent breeding with wild-type animals results in germ line transmission of the ESC-derived genome at variable frequencies (often equal to zero). Because the efficiency of gene transfer is low and because large numbers of recipient animals are required for embryo transfer, production of transgenic large animals by this method has been difficult. ESCs have also been used to specifically integrate a transgene to a given locus of a genome using homologous recombination for the production of knock-out animals. The process is very expensive and lengthy, sometimes requiring several back-crosses of the chimeric founders before a true breeding individual is obtained. The method is also limited to animals for which germline contributing ESC lines exist, namely the mouse.

Sperm-mediated DNA transfer to offspring has the potential to simplify the generation of transgenic animals. In particular, Intra Cytoplasmic Sperm Injection (ICSI; see U.S. Pat. No. 6,376,743) has resulted in the production of transgenic animals in a single step, resulting in savings, both of cost and time. The methods themselves, however, remain inefficient with ICSI-mediated transgenesis having a success rate in mice of 20% of the born offspring being transgenic. The method also has the disadvantage in relying on the need to freeze and therefore kill sperm in order to render them “sticky” to exogenous transgene linear double-stranded DNA. The procedure often results in transient gene expression, and the freezing of sperm may result in chromosomal breaks, hindering the development of transgenic embryos and reducing birth rates (Szczygiel et al., 2003, Biol. Reprod. 68(5):1903-10). It is therefore necessary to use approximately 100 oocytes to obtain 2 transgenic mice out of 14 live born pups.

Transgenic animals are important for scientific, pharmaceutical and agricultural purposes. Production of foreign proteins in milk using genetically engineered livestock has been suggested as a system for making therapeutic recombinant proteins. Moreover, the insertion of human genes into the genome of animals, such as pigs could allow such animals to act as living organ or cell factories for human organs or cells tolerated by the human body. Although transgenesis is relatively easy in the mouse, similar levels of success are not easy to achieve in larger animals. Livestock usually have long gestation periods compared to the mouse gestation of twenty-one days and have a need for a greater number of surrogate mothers for the production of the same number of animals obtained in mouse transgenesis. The expense and time involved in producing transgenic animals by current methods becomes a prohibitively daunting process for the livestock industry.

Therefore there remains a need to improve the efficiency of gene transfer strategies to produce transgenic animals reliably and this invention meets that need.

### SUMMARY OF THE INVENTION

The present invention provides methods for producing transgenic embryos and animals with a higher efficiency than presently available procedures based on Intracytoplasmic Sperm Injection (ICSI) of nucleic acid complexed with recombinase.

Therefore, in one aspect of the invention, a method for obtaining a transgenic embryo is provided, comprising the steps of: (a) incubating an exogenous nucleic acid with a recombinase; (b) introducing the exogenous nucleic acid and the recombinase into an unfertilized oocyte; (c) introducing a spermatozoon or a sperm head into the unfertilized oocyte (e.g., a metaphase II oocyte) to form a transgenic fertilized oocyte; and (d) allowing the transgenic fertilized oocyte to develop into a transgenic embryo.

In one embodiment step (b) and step (c) are carried out simultaneously, for example by a single microinjection into the cytoplasm of the unfertilized oocyte or in combination with in vitro fertilization.

In another embodiment live spermatozoon is utilized in the method either directly or in the production of sperm heads. Herein the terms “live spermatozoon” and “fresh spermatozoon” are used interchangeably.

The exogenous nucleic acid can be any nucleotide sequence of interest, such as a sequence derived from a human gene sequence, a nucleic acid comprising one or more structural gene sequences, optionally with operably linked regulatory nucleic acid sequences, such as a constitutive, tissue-specific or inducible promoter. Useful structural gene sequence include sequences encoding a polypeptides, such as receptors, enzymes, cytokines, hormones, growth factors, immunoglobulins, cell cycle proteins, cell signaling proteins, membrane proteins, cytoskeletal proteins and reporter proteins (e.g., green fluorescent protein, beta-galactosidase, alkaline phosphatase or luciferase). Reporter proteins are particularly useful to monitor the development of a cell or tissue in a transgenic embryo. Alternatively, the structural gene sequence may encode a ribonucleic acid, such as a regulatory RNA, an antisense nucleic acid, a ribozyme, dsRNA or a small interfering ribonucleic acid (siRNA). The structural gene sequence includes disease genes, for example, where the disease gene is linked to cardiovascular diseases, neurological diseases, reproductive disorders, cancers, eye diseases, endocrine disorders, pulmonary diseases, metabolic disorders, hereditary diseases, autoimmune disorders and/or aging.

The oocyte and the spermatozoon or sperm head may be from a mammal, for example non-human primates, ovines, bovines, porcines, ursines, felines, canines, equines and rodents. Alternatively, the oocyte and the spermatozoan or sperm head may be from an invertebrate, a fish, an amphibian, a reptile, a bird, a sea urchin, a lobster, an abalone, or a shellfish.

The methods of the invention may include the step of allowing the transgenic embryo to develop into a live offspring, for example by transplanting the transgenic embryo into a surrogate mother to produce a transgenic animal.

The transgenic animal can be useful as a model for human disease, such as cardiovascular diseases, neurological diseases, reproductive disorders, cancers, eye diseases, endocrine disorders, pulmonary diseases, metabolic disorders, autoimmune disorders and/or aging, or a hereditary disease. The transgenic animal can be useful as a model for embryo and fetal development, as a model to demonstrate the safety and efficacy of one or more treatments, such as drug therapy, gene therapy, stem cell therapy and/or somatic cell therapy, or as a model for disease diagnosis.

Also provided by the invention are the transgenic embryos, transgenic stem cells and the resulting transgenic animals produced according to the methods of the invention.

In a further aspect of the invention, there is provided a method of producing transgenic primate cells, comprising the steps of: (a) incubating an exogenous nucleic acid with a recombinase; (b) introducing the exogenous nucleic acid and the recombinase into an unfertilized primate oocyte; (c) introducing a spermatozoon or a sperm head of the same species into said unfertilized oocyte to form a transgenic fertilized oocyte; and (d) allowing the transgenic fertilized oocyte to develop into transgenic cells suitable for use in treating human diseases. Such human diseases include cardiovascular disease, neurological diseases, reproductive disorders, cancer, eye diseases, endocrine disorders, pulmonary disease, metabolic disorders, autoimmune disorders, and/or conditions related to aging.

Also provided by the invention are compositions and kits comprising (a) a recombinase; (b) an exogenous nucleic acid; and (c) a spermatozoon and/or sperm head.

### DETAILED DESCRIPTION OF THE INVENTION

Definitions

For convenience, the meaning of certain terms and phrases employed in the specification, examples, and appended claims are provided below.

As used herein, the term “egg” when used in reference to a mammalian egg, means an oocyte surrounded by a zona pellucida and a mass of cumulus cells (follicle cells) with their associated proteoglycan. The term “oocyte” refers to a female gamete cell and includes primary oocytes, secondary oocytes and mature, unfertilized ovum. An oocyte is a large cell having a large nucleus (i.e., the germinal vesicle) surrounded by ooplasm. The ooplasm contains non-nuclear cytoplasmic contents including mRNA, ribosomes, mitochondria and yolk proteins.

The term “unfertilized oocyte” as used herein refers to any female gamete cell which has not been fertilized and encompasses both pre-maturation and pre-fertilization oocytes.

The term “prefertilization oocyte” as used herein refers to a female gamete cell such as a pre-maturation oocyte following exposure to maturation medium in vitro but prior to exposure to spermatozoan or sperm head (i.e., matured but not fertilized). The prefertilization oocyte has completed the first meiotic division, has released the first polar body and lacks a nuclear membrane (the nuclear membrane will not reform until fertilization occurs). After fertilization, the second meiotic division occurs along with the extrusion of the second polar body and the formation of the male and female pronuclei). Prefertilization oocytes may also be referred to as matured oocytes at metaphase II of the second meiosis.

The term “sperm” refers to a male gamete cell and includes spermatogonia, primary spermatocytes, secondary spermatocytes, spermatids, differentiating spermatids, round spermatids, and spermatozoa. The term “sperm head” refers to a manipulated sperm whereby the tail portion has been removed.

The term “somatic cell” refers to any animal cell other than a germ cell or germ cell precursor.

The term “embryonic stem cell” or “stem cell” refers to a cell, which is an undifferentiated cell and may undergo terminal differentiation giving rise to many differentiated cell types in an embryo or adult, including the germ cells (sperm and eggs). This cell type is also referred to herein as an “ESC”.

The term “animal” includes all vertebrate animals such as mammals (e.g., rodents, sheep, dogs, cows, pigs and primates, including monkeys, apes, and humans), amphibians, reptiles, fish, birds and Invertebrates. It also includes an individual animal in all stages of development, including embryonic and fetal stages.

A “transgenic animal” refers to any animal, preferably a mammal (e.g., mouse, rat, squirrel, hamster, guinea pig, pig, baboons, squirrel monkey, and chimpanzee, etc.), bird or an amphibian, in which one or more cells contain exogenous nucleic acid introduced by way of human intervention. The transgene is introduced into the cell, directly or indirectly, by introduction into a precursor of the cell, by way of deliberate genetic manipulation. In the transgenic animals described herein, the transgene can cause cells to express a structural gene of interest. However, transgenic animals in which a transgene is silent are also included.

The term “transgenic embryo” refers to an embryo containing a transgene.

The term “transgenic cell” refers to a cell containing a transgene.

The term “germ cell line transgenic animal or embryo” refers to a transgenic animal or embryo in which the genetic alteration or genetic information was introduced into a germ line cell, thereby conferring the ability to transfer the genetic information to offspring. If such offspring possess some or all of that alteration of genetic information, they are transgenic animals as well.

The term “exogenous nucleic acid,” refers to nucleic acid that is not naturally present in the cell, oocyte, sperm head or spermatozoan or a nucleic acid which is present in a position other than its naturally occurring position in the cell, oocyte, sperm head or spermatozoan.

The term “gene” refers to a nucleic acid that comprises control and structural (e.g. coding) sequences necessary for the production of a ribonucleic acid and/or a polypeptide. The polypeptide can be encoded by a full length coding sequence or by any portion of the coding sequence so long as the desired function (or lack of function) is obtained.

The term “transgene” broadly refers to any nucleic acid that is introduced into the genome of an animal or embryo, including but not limited to genes, fragments thereof (e.g., regulatory sequences or structural sequences) or DNA having sequences which are perhaps not normally present in the genome, genes which are present, but not normally transcribed and/or translated (“expressed”) in a given genome, or any other gene or DNA which one desires to introduce into the genome. This may include genes which may normally be present in the no transgenic genome but which one desires to have altered in expression, or which one desires to introduce in an altered or variant form. The transgene may also be foreign to the nontransgenic genome (e.g., reporter genes or DNA encoding hairpin siRNAs). The transgene may be specifically targeted to a defined genetic locus or may be randomly integrated within a chromosome. A transgene may therefore include one or more transcriptional regulatory sequences and any other nucleic acid, such as introns, that may be necessary for optimal expression of a selected nucleic acid. A transgene can be as few as a 100-150 bp of nucleotides long, but is preferably at least about 200, 250, 300, 350, 400, or 500 nucleotides long and longer. A transgene can be coding or non-coding sequences, or a combination thereof. A transgene usually comprises a regulatory element that is capable of driving the expression of one or more nucleic acids under appropriate conditions.

The term “a structural gene” refers to a gene that expresses a biologically active protein of interest or a ribonucleic acid, such as an antisense RNA, ribozyme or siRNA. The term “structural gene” excludes the non-coding regulatory sequence which drives transcription. The structural gene may be derived in whole or in part from any source known to the art, including from a eukaryotic, prokaryotic, human, animal, plant, fungal, yeast, insect, viral or other source, or chemically synthesized. The structural gene may therefore also encode a fusion protein. A structural gene may contain one or more modifications in either the coding or the untranslated regions which could affect the biological activity or the chemical structure of the expression product, the rate of expression, or the manner of expression control. Such modifications include, but are not limited to, mutations, insertions, deletions, and substitutions of one or more nucleotides as is apparent to the skilled person. The structural gene may constitute an uninterrupted coding sequence or it may include one or more introns, bound by the appropriate splice junctions.

The term “genome” is intended to include the entire DNA complement of an organism, including the nuclear DNA component, chromosomal or extrachromosomal DNA, as well as the cytoplasmic domain (e.g., mitochondrial DNA).

The term “transgene construct” refers to a nucleic acid molecule, (e.g., vector), which contains a structural gene of interest that has been generated for the purpose of the expression of a specific nucleotide sequence(s), or is to be used in the construction of other recombinant nucleotide sequences.

As used herein, the term “vector” refers to a nucleic acid molecule capable of transporting another nucleic acid to which it has been linked. Preferred vectors are those capable of autonomous replication and/expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer to circular double-stranded DNA that in their vector form are not bound to the chromosome. In the present specification, “plasmid” and “vector” are used interchangeably as the plasmid is the most commonly used form of vector. However, the invention is intended to include such other forms of expression vectors that serve equivalent functions.

The term “gene expression” refers to the process by which a nucleotide sequence undergoes successful transcription and, for polypeptides, translation such that detectable levels of the delivered nucleotide sequence are expressed.

The term “promoter” refers to the minimal nucleotide sequence sufficient to direct transcription. Promoter elements may render promoter-dependent gene expression controllable for cell-type specific, tissue specific, or inducible by external signals or agents. Such elements are usually located in the 5′ region of the gene but may also be located in the coding, non-coding or 3′ regions of the gene. The term “inducible promoter” refers to a promoter where the rate of RNA polymerase binding and initiation of transcription can be modulated by external or internal stimuli. The term “constitutive promoter” refers to a promoter where the rate of RNA polymerase binding and initiation of transcription is constant and relatively independent of external or internal stimuli. A “temporally regulated promoter” is a promoter where the rate of RNA polymerase binding and initiation of transcription is modulated at a specific time during development.

As used herein, the term “regulatory nucleic acid sequence” refers to a nucleic acid sequence capable of controlling the transcription of an operably associated structural gene. A regulatory sequence of the invention may include a promoter, an enhancer, and/or a silencer. Therefore, placing a structural gene under the regulatory control of a promoter or a regulatory element means positioning the structural gene such that the expression of the structural gene is controlled by the regulatory sequence(s). In general, promoters are found positioned 5′ (upstream) of the genes that they control. Thus, in the construction of promoter—structural gene combinations, the promoter is preferably positioned upstream of the structural gene and at a distance from the transcription start site that approximates the distance between the promoter and the structural gene it controls in the natural setting. As is known in the art, some variation in this distance can be tolerated without loss of promoter function. Similarly, the preferred positioning of a regulatory element, such as an enhancer, with respect to a structural gene placed under its control reflects its natural position relative to the structural gene it naturally regulates. Enhancers are believed to be relatively position and orientation independent in contrast to promoter elements. In addition, 3′ untranslated regions such as polyA signals may also be utilized as a regulatory sequence.

The term “recombinase” as used herein refers to RecA-like proteins and may be present with other subunits of a recombinase complex. Recombinases may be characterized by forming a filament with ssDNA. They typically induce strand exchange in providing natural strand displacement by the incoming strand, and are normally associated with DNA repair. Various naturally occurring or mutant recombinases are available, particularly recA from E. coli, e.g. recA-803, Rad 51 and Rad 52 from S. cerevisiae, Rad 51-like, DMC1, mei3 from N. crassa, as well as human recombinase from human cells.

The term “in vitro fertilization” refers to the mixing of live, intact spermatozoa with oocytes to form embryos.

The term “ICSI” or “intracytoplasmic sperm injection” refers to the microinjection of sperm heads into the cytoplasm of an oocyte to achieve fertilization and to allow the subsequent formation of embryos.

The term “antisense nucleic acid” refers to nucleic acid molecules that are complementary to, or that hybridize to, at least a portion of a specific nucleic acid molecule, such as an RNA molecule (e.g., an mRNA molecule). The antisense nucleic acids hybridize to corresponding nucleic acids, such as mRNAs, to form a double-stranded molecule, which interferes with translation of the mRNA, as the cell will not translate a double-stranded mRNA. Antisense nucleic acids encoded by the nucleic acids used in the invention are typically at least 10-12 nucleotides in length, for example, at least 15, 20, 25, 50, 75, or 100 nucleotides in length. The antisense nucleic acid can also be as long as the target nucleic acid with which it is intended to form an inhibitory duplex. The antisense nucleic acids are produced in the transgenic cell in which a nucleic acid encoding the antisense nucleic acid has been introduced.

The term “ribozyme” refers to an enzymatic nucleic acid molecule that catalyzes one or more of a variety of cleaving reactions, including the ability to repeatedly cleave other separate nucleic acid molecules in a nucleotide base sequence-specific manner. Such enzymatic nucleic acid molecules can be used, for example, to target virtually any RNA transcript (Zaug et al., 324, Nature 429 1986; Cech, 260 JAMA 3030, 1988; and Jefferies et al., 17 Nucleic Acids Research 1371, 1989).

The term “short interfering RNA” (or siRNA) refers to RNA molecules of at least partially double stranded character of about 21-23 nt length (and therefore includes hairpin structures), preferably with 5′ terminal phosphate and 3′ short overhangs (˜2 nt). The siRNAs target the corresponding mRNA sequence specifically for destruction (Zamore P. D. Nature Structural Biology, 8, 9, 746-750, (2001).

The methods of the present invention provide innovative and powerful approaches for routinely producing transgenic nonhuman primate specimens for clinically relevant research and for creating transgenic primates for diagnosing, preventing, and curing human diseases. The present inventors describe for the first time the efficient use of ICSI-Transgenesis (ICSI-Tr) using live spermatozoa instead of freeze-thawed or similarly pre-treated spermatozoa to obtain transgenesis. Freeze-thawed spermatozoa exhibit chromosomal aberrations, which contribute to the low efficiency of transgenesis. However, pre-treated spermatozoa can bind exogenous, linear dsDNA typically used for transgenesis more efficiently than live spermatozoa, which has meant until now that ICSI has been carried out using freeze-thawed spermatozoa with acceptance of its disadvantages.

The present inventors have made use of the easily available bacterial recombinase to obtain transgene integration events in early mouse embryos. In the presence of RecA the interaction of ssDNA with live sperm was strong and led to transgenic embryos after ICSI. When a pre assembled RecA and ssDNA complex was co-injected with fresh sperm head into MII oocytes, transgene expression (exemplified using green fluorescent protein expression) was observed in embryos as soon as at the two cell stage, and was maintained during the subsequent developmental stages. The fluorescence detected in early embryos might have been a result of transient expression. However, the majority of green progeny produced was truly transgenic, and were able to transmit their transgene to offspring, with only one mouse showing chimeric EGFP expression. This single chimeric animal was also able to transmit its transgene to offspring indicating that the transgene was expressed in germ line cells.

Thus, the present invention provides a method for obtaining a transgenic embryo, comprising the steps of:
(a) incubating an exogenous nucleic acid with a recombinase;
(b) introducing the exogenous nucleic acid and the recombinase into an unfertilized oocyte;
(c) introducing a spermatozoon or a sperm head into said unfertilized oocyte to form a transgenic fertilized oocyte; and
(d) allowing the transgenic fertilized oocyte to develop into a transgenic embryo.

The exogenous nucleic acid will be typically prepared by genetic manipulation of known sequences to provide a desired product. The product may result in an enhanced biological activity or reduced or abolished activity depending on the desired outcome. Sequences derived from a human origin are particularly useful for the therapeutic and diagnostic methods set out in more detail below.

The transgene may encode a polypeptide having the desired biological activity. Such polypeptides include without limitation receptors, enzymes, cytokines, hormones, growth factors, immunoglobulins, cell cycle proteins, cell signaling proteins, membrane proteins and cytoskeletal proteins. The exogenous nucleic acid may include a structural gene sequence associated with a disease, such as a disease gene linked to cardiovascular disease, neurological disease, reproductive disorders, cancer, eye disease, endocrine disorders, pulmonary disease, metabolic disorders, hereditary disease, autoimmune disorders and/or aging. Introduction of a mutant gene may also be useful for preparing cellular or animal models reflecting a chosen disease.

The structural gene may be a reporter gene, which can be used effectively in model systems and as a powerful tool for determining successful delivery of exogenous DNA into a target cell. Many transgenic reporters are available but the most commonly and widely used is green fluorescent protein (GFP) which has been used in many applications including developmental and basic biological studies (Naylor, 58 BIOCHEM. PHARMACOL. 749-57, 1999; Ikawa et al., 430 FEBS LETT. 83-87, 1998; Rizzuto et al., 6 CURR. BIOL. 183-88, 1996). Other transgenic reporters include, but are not limited to, beta-galactosidase, luciferase, and secreted placental alkaline phosphatase. The enzyme beta-galactosidase, catalyzes the hydrolysis of molecules containing beta-gal linkages and the reaction product can be detected by a colorimetric assay (Kubisch et al., 104 J. REPROD. FERTIL. 133-39, 1995; Chan et al., 52 MOL. REPROD. DEV. 406-13, 1999). Luciferase catalyzes the oxidative decarboxylation of luciferin producing a yellow-green light and its activity may be detected by photon imaging (Thompson et al., 92 PROC. NATL. ACAD. SCI. USA 1317-21, 1995; Menck et al., 7 TRANSGENIC RES. 331-41, 1998). Secreted placental alkaline phosphatase (SEAP), a truncated form of placental alkaline phosphatase, is constitutively secreted and can be detected by chemiluminescence (Chan et al., 52 BIOL. REPROD. 137, 1995).

Alternatively, the transgene may encode one or more antisense nucleic acids. For example, an antisense polynucleotide sequence (complementary to the DNA coding strand) may be introduced into the oocyte to decrease the expression of a “normal” gene. This approach utilizes, for example, antisense nucleic acid, ribozymes, triplex agents or siRNA to block transcription or translation of a specific mRNA, either by masking that mRNA with an antisense nucleic acid or triplex agent, or by cleaving it with a ribozyme.

Double stranded RNA (dsRNA) has been found to be even more effective in gene silencing than antisense alone (Fire A. et al., Nature, Vol 391, (1998)). DsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245).

RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction (Zamore P. D. Nature Structural Biology, 8, 9, 746-750, (2001). Thus in one embodiment, inhibition of a desired gene is achieved using double stranded RNA comprising a fragment of a coding sequence, in particular a sequence selective for the particular gene of interest, which may for example be a double stranded RNA (which will be processed to siRNA, as described above). Cellular defense mechanisms, such as the PKR pathway will typically need to be circumvented in mammalian systems, for example using siRNA directed against the individual components. These RNA products may be synthesized in vivo by the transgene.

However, to avoid the PKR pathway, siRNA duplexes of about 21-23 nucleotides in length with 3′-overhang ends are preferably used (Zamore P D et al., Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)). Thus, siRNA duplexes of between 20 and 25 bps, more preferably between 21 and 23 bps, of the desired sequence, in particular sequences selective for the desired gene sequence either by synthesis of two separate strands or as a hairpin can be expressed by the transgene(s).

Where one or more structural genes (e.g., encoding a protein) are used as transgenes, it may be desirable to operably link the gene to an appropriate regulatory nucleic acid sequences, which will allow expression of the transgene. A transgene construct may therefore comprise a nucleic acid sequence in both the sense and antisense orientation, such that when expressed as RNA the sense and antisense sections will associate to form a double stranded RNA. This may for example be a long double stranded RNA (e.g., more than 23 nts) or siRNA hairpin structures. Alternatively, the sense and antisense sequences are provided on different vectors. These vectors and RNA products are useful, for example, to inhibit de novo production of a desired polypeptide in a cell. Targeted expression of transgenes can be achieved using tissue-specific promoters, such as promoters specific for adipose tissue, muscle, liver or other tissues.

Regulatory elements, e.g., promoters, enhancers, (e.g., inducible, tissue-specific or constitutive), or polyadenylation signals are well known in the art. Regulatory sequences can be endogenous regulatory sequences, i.e., regulatory sequences from the same animal species as that in which it is introduced as a transgene. The regulatory sequences can also be the natural regulatory sequence of the gene that is used as a transgene. A controllable promoter system or gene expression system is the most desirable. The choice of stage specific and/or a tissue specific promoter depends on the gene or target organ of interest.

For a gene delivery system, the strong viral promoter, cytomegalovirus (CMV), is a suitable promoter as well as the protamine-1 promoter (O'Gorman et al., 94 PROC. NATL. ACAD. Sci. USA 14602-07, 1997). This promoter has been widely used in transgenic studies. Although it lacks specificity, its constitutive expression pattern will be an advantage during evaluation of gene delivery efficiency.

Other useful promoters for gene expression regulation include, but are not limited to, promoters for genes derived from viruses (e.g., Moloney leukemia virus), and promoters for genes derived from various mammals (e.g., humans, pigs, rabbits, dogs, cats, guinea pigs, hamsters, rats, and mice). Preferred promoters are those from the structural gene of interest (e.g., genes for insulin, erythropoietin, or platelet-derived growth factor). In another preferred embodiment, inducible promoters (e.g., tetracycline regulation system, metallothionein promoter or hormone-inducible promoters) may be utilized to regulate the expression of the transgene (lida et al., 70 J. VIROL. 6054-59,1996; Palmiter, 91 PROC. NATL. ACAD. Sci. USA 1219-23, 1994).

A transgene construct as described herein may also include a 3′ untranslated region downstream of the DNA sequence. Such regions can stabilize the RNA transcript of the expression system and thus increase the yield of desired product from the expression system. Among the 3′ untranslated regions useful in the constructs of this invention are sequences that provide a polyA signal. Such sequences may be derived, e.g., from the SV40 small t antigen, or other 3′ untranslated sequences well known in the art. The length of the 3′ untranslated region is not critical but the effect of its polyA transcript appears important in stabilizing the mRNA of the expression sequence.

A transgene construct may also include a 5′ untranslated region between the promoter and the DNA sequence encoding the signal sequence. Such untranslated regions can be from the same control region from which promoter is taken or can be from a different gene, e.g., they may be derived from other synthetic, semi-synthetic, or natural sources.

The transgene construct preferably allows the production of single stranded DNA for introduction into the oocyte as is exemplified below.

The transgene constructs described herein may be inserted into any suitable plasmid, bacteriophage, or viral vector for amplification, and may thereby be propagated using methods known in the art, such as those described by Maniatis et al. (MOLECULAR CLONING: A LABORATORY MANUAL, Cold Spring Harbor, N.Y., 2000). A construct may be prepared as part of a larger plasmid, which allows the cloning and selection of the constructs in an efficient manner as is known in the art. Constructs may be located between convenient restriction sites on the plasmid so that they may be easily isolated from the remaining plasmid sequences for incorporation into the desired mammal.

Preferably, the endogenous nucleic acid is a single-stranded DNA, in which case the transgene construct can be inserted into vectors such as M13, allowing the synthesis of large amounts of the desired DNA. Alternatively, other techniques for synthesizing single-stranded DNA are well known in the art, such as chemical synthesis or PCR.

Although the exogenous nucleic acid and recombinase could form a complex within the oocyte without prior contact, typically the exogenous nucleic acid is incubated with a recombinase prior to introduction into the cell according to the methods of the invention. Coating the nucleic acid with a recombinase typically produces a nucleoprotein filament, and is achieved by combining the nucleic acid, preferably a single-stranded DNA with the recombinase in an appropriate buffered medium in the presence of a cofactor and magnesium ion. A variety of cofactors may be employed, such as ATPγS, rATP, dATP, GTPγS, or equivalent molecules, mixtures thereof, and the like. The ratio of the recombinase per nucleotide of the probe will generally range from about 1-80:1 usually to 2-40:1. Thus, a high concentration of recombinase and cofactor may be employed to provide for a substantially complete coating of nucleic acid. Alternatively, lower amounts of the recombinase may be employed where a lower ratio of recombinase to nucleotide will suffice. Generally, the cofactor will be in the range of about 0.2-12 mM, preferably about 2.4-8 mM. The magnesium ion will generally range from about 4-25 mM, more usually 6-8 mM, and may be conveniently present as an acetate or chloride. The time period of the incubating step is typically about 30 seconds to about 5-60 minutes. The complex may then be isolated and purified by any convenient means, if desired but is not necessary to practice the invention. Conveniently, the nucleic acid may be isolated by filtration, where the filter will retain the recombinase coated nucleic acid, while allowing individual molecules to pass through the filter, by gravity sedimentation, centrifugation, or the like.

The present inventors exemplify the invention below using the RecA gene product during ICSI mediated transgenesis. RecA is the bacterial homologue of the mammalian recombinase protein Rad51 which catalyses DNA repair and homologous recombination in mammalian cells. Any recombinase may be used in the practice of the present invention, including various naturally occurring or mutant recombinases, particularly RecA from E. coli, e.g. recA-803, Rad 51 and Rad 52 from S. cerevisiae, Rad 51-like, DMC1, mei3 from N. crassa, as well as human recombinase from human cells.

One or more transgenes and the recombinase can be efficiently introduced into a host genome using the methods of the present invention. A single microinjection into the cytoplasm of the unfertilized oocyte can be carried out with the recombinase complexed to the exogenous nucleic acid, preferably single-stranded DNA, together with a sperm head obtained from a fresh spermatozoon, thereby providing a simple, yet effective procedure.

The role of spermatozoa or the sperm head during fertilization involves the transfer of a haploid genome to the resultant zygote. This capacity has been exploited for the delivery of exogenous DNA for the production of transgenic animals (Lauria & Gandolfi, 36 MOL. REPROD. DEV. 255-57, 1993; Kim et al., 46 MOL. REPROD. DEV. 1-12, 1997; Chan et al., MOL. HUMAN REPROD. 26-33, 2000; Perry et al., 284 SCIENCE 1180-83, 1999; U.S. Pat. No. 6,376,743(ICSI); Lavitrano et al., 57 CELL 717-23, 1989), the teachings of which are incorporated herein by reference.

The oocyte and the spermatozoon or sperm head can be from any compatible source, such as both being from a mammalian source of the same species, or from an invertebrate, a fish, an amphibian, a reptile or a bird, a sea urchin, a lobster, an abalone or a shellfish. The mammal can be a non-human primate, ovine, bovine, porcine, ursine, feline, canine, equine or rodent.

Generally, the transgene is introduced by microinjection and the fertilized oocytes are then cultured in vitro until a pre-implantation embryo is obtained preferably containing about 16-150 cells (see e.g., U.S. Pat. No. 4,873,191). Methods for culturing fertilized oocytes to the pre-implantation stage are described by Gurdon et al. (101 METH. ENZYMOL. 370-86, 1984); HOGAN ET AL. (MANIPULATION OF THE MOUSE EMBRYO: A LABORATORY MANUAL, C.S.H.L. N.Y., 1986); Hammer et al. (315 NATURE 680-83, 1985); Gandolfi et al. (81 J. REPROD. FERT. 23-28, 1987); Rexroad et al. (66 J. ANIM. Sci. 947-953, 1988); Eyestone et al. (85 J. REPROD. FERT. 715-720, 1989); and Camous et al. (72 J. REPROD. FERT. 779-785, 1984). The pre-implantation embryos may be frozen pending implantation. Pre-implantation embryos are transferred to the oviduct of a pseudopregnant female resulting in the birth of a transgenic or occasionally a chimeric animal, depending upon the stage of development when the transgene is integrated. Chimeric mammals can be bred to form true germline transgenic animals. Thus the invention provides a method for producing transgenic animals, preferably nonhuman primates, further comprising the step of allowing the transgenic embryo to develop into a live offspring. This may be achieved by transplanting the transgenic embryo into a surrogate mother to produce a transgenic animal. The resulting transgenic animal can be useful as a model for human disease, such as a model for one or more of cardiovascular diseases, neurological diseases, reproductive disorders, cancers, eye diseases, endocrine disorders, pulmonary diseases, metabolic disorders, autoimmune disorders and aging. The transgenic animal may be useful as a model for hereditary disease, for embryo and fetal development, for disease diagnosis, or to demonstrate the safety and efficacy of drug therapy, gene therapy, stem cell therapy or somatic cell therapy. Also provided are the transgenic animals obtained by the methods of the invention.

Transgenic offspring may be detected by any of several means well known to those skilled in the art. Non-limiting examples include Southern blot or Northern blot analyses, using a probe that is complementary to at least a portion of the transgene. Western blot analysis using an antibody against the protein encoded by the transgene may be employed as an alternative or additional method for screening for the presence of the transgene product. A DNA sample may be prepared from a tissue or cell and analyzed by PCR for expression of the transgene.

Alternative or additional methods for evaluating the presence of the transgene include, without limitation, biochemical assays such as enzyme and/or immunological assays, histological stains for a particular marker or enzyme activity, flow cytometric analysis, in situ hybridization of mRNA analysis, and FACS analysis of protein expression. Analysis of the blood may also be useful to detect the presence of the transgene product in the blood, as well as to evaluate the effect of the transgene on the levels of various types of blood cells and other blood constituents.

Animal tissue may also be analyzed directly, for example, by preparing tissue sections. In some embodiments, it may be preferable to fix the tissue (e.g., with paraformaldehyde or formalin). Tissue sections may be prepared frozen, or may be paraffin-embedded. Slides of animal tissue may be used for immunohistochemistry, in vitro hybridization or histology (e.g., hematoxylin and eosin staining).

Transgenic cells, genetically identical cells, and stem cells derived from primates are invaluable for the study of numerous diseases (e.g., aging, AIDS, cancer, Alzheimer's disease, autoimmune diseases, metabolic disorders, obesity, organogenesis, psychiatric illnesses, and reproduction). Furthermore, the importance of these cells for molecular medicine and the development of innovative strategies for gene therapy protocols should not be minimized. For example, clinical strategies may include assisted reproductive technologies, transgenesis, and use of totipotent and immortalized embryonic germ (EG) and stem cells (ES). In addition, identical, transgenic and/or immortalized, totipotent EG or ES-derived cells may be ideal preclinical models in identifying the molecular events related to infertility, gametogenesis, contraception, assisted reproduction, the genetic basis of infertility, male versus female meiotic cell cycle regulation, reproductive aging, and the non-endocrine basis of idiopathic infertility.

Transgenesis may also be used to discover disease mechanisms and to create and optimize molecular medical cures. For example, monkeys created with a genetic knockout for a specific gene may accelerate discovery of the cures for cancer, arteriosclerosis causing heart disease and strokes, inborn errors of metabolism and other fetal and neonatal diseases, Parkinson's disease, polycystic kidney disease, blindness, deafness, sensory disorders, storage diseases (Lesch-Nyan and Zellwegers) and cystic fibrosis. These transgenic animals may also be amenable for evaluating and improving cell therapies including diabetes, liver damage, kidney disease, artificial organ development, wound healing, damage from heart attacks, brain damage following strokes, spinal cord injuries, memory loss, prion infection, Alzheimer's disease and other dementia, muscle and nerve damage.

Thus, the present invention also relates to methods of preparing and using transgenic embryonic cells, in particular to treat human diseases. Specifically, the methods to produce transgenic animals and transgenic primates, described in the present invention, may also be used to create transgenic embryonic stem cells.

Briefly, following fertilization, an egg divides over a period of days to form a blastocyst which, generally, is a hollow ball of cells having an inner cell mass and a fluid-filled cavity, both encapsulated by a layer of trophoblast cells. Cells from the inner cell mass of an embryo (i.e., blastocyst) may be used to derive a cell line referred to as embryonic stem cells (ESCs), and these cells may be maintained in tissue culture (see e.g., Schuldiner et al., 97 PROC. NATL. ACAD. Sci. USA 11307-12, 2000; Amit et al., 15 DEV. BIOL. 271-78, 2000; U.S. Pat. Nos. 5,843,789, 5,874,301). In general, stems cells are relatively undifferentiated, but may give rise to differentiated, functional cells. For example, hematopoietic stem cells may give rise to terminally differentiated blood cells such as erythrocytes and leukocytes.

Thus, in a further aspect of the invention, a method of producing transgenic primate cells, comprising the steps of:
(a) incubating an exogenous nucleic acid with a recombinase;
(b) introducing the exogenous nucleic acid and the recombinase into an unfertilized primate oocyte;
(c) introducing a spermatozoon or a sperm head of the same species into said unfertilized oocyte to form a transgenic fertilized oocyte; and
(d) allowing the transgenic fertilized oocyte to develop into transgenic cells suitable for use in treating human diseases.

Using the methods provided by the present invention, transgenic primate embryonic stem cells may be produced which express a gene related to a particular disease, e.g., cardiovascular disease, neurological diseases, reproductive disorders, cancer, eye diseases, endocrine disorders, pulmonary disease, metabolic disorders, autoimmune disorders or aging. For example, transgenic primate embryonic cells may be engineered to express tyrosine hydroxylase which is an enzyme involved in the biosynthetic pathway of dopamine. In Parkinson's disease, this neurotransmitter is depleted in the basal ganglia region of the brain. Thus, transgenic primate embryonic cells expressing tyrosine hydroxylase may be grafted into the region of the basal ganglia of a patient suffering from Parkinson's disease and potentially restore the neural levels of dopamine (see e.g., Bankiewiez et al., 144 Exp. NEUROL. 147-56, 1997). The methods described in the present invention, therefore, may be used to treat numerous human diseases (see e.g., Rathjen et al., 10 REPROD. FERTIL. DEV. 31-47, 1998; Guan et al., 16 ALTEX 135-41, 1999; Rovira et al., 96 BLOOD 4111-117, 2000; Muller et al., 14 FASEB J. 2540-48, 2000).

Transgenic “knock out” animals are also provided by the present invention by using a transgene with a mutation in a gene or resulting in deletion of at least a portion of a gene. Such knock-out animals are useful as models for the specific human disease resulting from the loss in the specific gene function.

### EXAMPLES

The examples are described for the purposes of illustration and are not intended to limit the scope of the invention. Variations in the design and detection of the assays will be apparent to one of ordinary skill in the art.

Methods of molecular genetics, protein and peptide biochemistry and immunology referred to but not explicitly described in this disclosure and examples are reported in the scientific literature and are well known to those skilled in the art. For example, standard methods in genetic engineering are carried out essentially as described in Sambrook et al., Molecular Cloning: A laboratory manual, 2nd Ed., Cold Spring Harbor Laboratory Press, Cold Spring Harbor N.Y., 2000.

### Example 1

### ICSI-Mediated Transgenesis Using Live Spermatozoa

This Example demonstrates that the recombinase protein RecA together with ssDNA introduced with live spermatozoa into mouse oocytes using ICSI is able to bring about efficient integration of an exogenous nucleic acid into the mouse genome. The method is illustrated using a reporter gene, namely green fluorescent protein.

Production of Single Stranded (ss) DNA

A plasmid, pCX-EGFP, is produced by inserting the EGFP gene downstream and operably linked to the cytomegalovirus enhancer, chicken beta-actin promoter and beta-actin intron, and upstream (i.e., followed at its 3′-end) of the bovine globin poly-adenylation signal (Ikawa M, et al., FEBS Lett 375 (1995) 125-128). The entire insert including the promoter, regulatory sequences and coding sequence is excised with the restriction enzymes Bam-HI and SalI and gel purified. The thus formed reporter cassette is then cloned into the polylinker site of an M13 phage-based vector (commercially available) flanked by two different Pvull restriction sites. Bacterial cells containing the F+-Pilus are transfected with the M13 phage-based vector and ssDNA phagemids are produced. After harvesting the ssDNA two different 30 mer oligonucleotides matching the sequence of the M13 phage flanking the reporter cassette and containing the Pvull restriction sites are annealed to the circular ssDNA, allowing digestion by Pvull. Typically, the excised ssDNA transfection cassette is gel purified using 0.8% low melting agarose, followed by AgarAce enzyme purification prior to use in ICSI-Tr.

Gel Retardation Assays

The gel purified ssDNA transgene cassette was quantitated and used for gel retardation experiments with the RecA protein to demonstrate the binding efficiency of the bacterial recombinase to ssDNA. Typically, RecA gel retardation assays were carried out.

Reaction mixtures (20.5 μL) comprised TKM buffer (25 mM Tris-HCl pH 8.0, 150 mM KCl, 2 mM MgCl₂) containing 10-25% glycerol ssDNA and RecA as set forth below, with and without ATP [1 mM].

| DNA (μg) | μL RecA (2 μg/μL) | TKM buffer, 10% glycerol (μL) |
|---|---|---|
| 0.5 | 0 | 20 |
| 0.5 | 5 | 15 |
| 0.5 | 10 | 10 |
| 0.5 | 20 | 0 |

The reactions were allowed to proceed on ice for one hour. The samples were mixed with 5 μL of Agarose—Blue loading dye and placed in the wells of a 4%, 1 mm thick acrylamide gel. The samples were then electrophoresed at 4° C. at 10V/cm gel until the loading dye reached the middle of the gel. The ssDNA was visualized by staining with SYBR Green II RNA dye.

RecA protein binds cooperatively to sequences of ssDNA with a stoichiometry of one RecA protein monomer for every three nucleotides of DNA. Gel retardation of ssDNA in the presence of RecA was more efficient in the absence of ATP than in its presence. It was necessary to use 80 μg of RecA protein to fully retard 1 μg of ssDNA in the absence of ATP. The DNA inside the RecA nucleoprotein filaments is completely protected against digestion by nucleases and phosphodiesterases.

In contrast to existing methods, this methodology advantageously allows the production of large quantities of ssDNA, which can be visualized without radioactive tagging. The use of linear ssDNA for transgenesis provides additional advantages over prior art methods where linear dsDNA is heat denatured prior to coating with RecA and pronulear microinjection.

### Example 2

### ICSI Injection of Transgenes into Oocytes

Preparation of media was as follows. CZB medium supplemented with 5.56 mM D-glucose was used for the culture of mouse oocytes after microinjection. The medium for oocyte collection and subsequent oocyte treatments, including micromanipulation, was a modified CZB medium (HEPES-CZB medium pH 7.4, containing 20 mM HEPES-HCl, 5 mM NaHCO₃ and 0.1 mg/mL polyvinyl alcohol (PVA; cold water soluble; Mr 30,000-70,000) instead of bovine serum albumin. CZB medium was used under 5% CO₂ in air, and HEPES-CZB was used under air.

Preparation of ssDNA+RecA mixture was as follows. If the final concentration of ssDNA required was 20 ng/μL, then a 40 ng/μL solution was prepared because of the 50% dilution necessitated during the mixing with spermatozoa. The ssDNA stock concentrations used ranged between 100 ng/μL and 300 ng/μL. The ssDNA and RecA incubation was done in a total of 20 μL on ice and was allowed to proceed for 1 h. Briefly: Add ssDNA volume to required concentration. Add 1×TKM buffer to required concentration. Incubate on a 95° C. heat block for 5 min. Quench on ice. Add RecA volume to required concentration. The RecA concentration used was either 2 μg/μL (New England Biolabs # M0249L, Beverly, Mass.) or 5 μg/μL (Epicentre # RC441 MG, Madison, Wis.). The 50% glycerol which RecA was shipped in was removed by preparing a Microspin G-25 column (Amersham Biosciences # 27-5325-01, Piscataway, N.J.) as follows: The column was washed by placing 70 μL of 1×TKM buffer into the center of the column bed and centrifuged at 735×g for 1 min at 4° C. Repeat the same centrifugation once more, and a third time with the duration of the spin lasting 2 min. Discard all flow-through solutions. Mix 35 μL of RecA with 35 μL of 2×TKM buffer, place to the center of the column bed and centrifuge for 2 min at 4° C. The 50% diluted RecA flow-through solution is free of glycerol. It is added to the ssDNA mixture at the required concentration to satisfy the 80:1 ratio in a total volume of 20 μL. The reaction mixture was incubated on ice for 1 h and then mixed with fresh sperm and used in transgenesis ICSI as described below. The ssDNA-RecA complex was typically preincubated with fresh spermatozoa before injection, as well as included in the injection buffer so that free RecA:ssDNA was also injected into the oocytes.

ICSI was carried out essentially as described by Kimura & Yanagimachi Biol Reprod 52: 709-720 (1995). Briefly, epididymal spermatozoa and matured oocytes were collected from 8-12 wk old B6D2F1 hybrid mice. Recipients of 2-cell embryos were 8-16 wk old random-bred CD-1 females. Oocytes were collected from superovulated B6D2F1 females induced by intraperitoneal injection of 5 IU pregnant mare serum gonadotropin (PMSG, Calbiochem, La Jolla, Calif.) followed by injection of 5 IU human chorionic gonadotrophin (hCG, Calbiochem, La Jolla, Calif.) 48 hours later. Matured oocytes were collected from the ampullary region of oviducts 13-15 h after hCG injection. Oocytes were freed from cumulus cells by treatment with 0.1% bovine testicular hyaluronidase (359 units/mg solid) in HEPES-CZB medium. Oocytes were rinsed and kept at room temperature (26° C.) in fresh HEPES-CZB medium before sperm injection.

Spermatozoa were collected by removing two epididymides from B6D2F1 males and a dense sperm mass was squeezed out of the caudae region of each epididymis after cutting it with a pair of sharp forceps. Sperm masses from two epididymides were gently placed at the bottom of 1 mL HEPES-CZB buffered solution in a microcentrifuge tube and kept at 37° C. for 10 min to allow spermatozoa to disperse into the solution. The upper 800 μL of the sperm suspension was collected and used for ICSI. The mixing of the ssDNA+RecA solution with sperm suspension was initiated by mixing 10 μL of each in a 20 μL final solution (i.e. If a 10 ng/μL solution of ssDNA+RecA concentration was required, then 10 μL of 20 ng/μL ssDNA+RecA solution was mixed with 10 μL of sperm suspension). Motile spermatozoa were collected and placed in another 20 μL droplet containing a mixture of the appropriate concentration of ssDNA+RecA solution and 12% (w/v) polyvinylpyrrolidone (PVP; Mr 360,000, ICN Pharmaceuticals, Costa Mesa, Calif.) prepared in HEPES-CZB. Therefore, if the final concentration of ssDNA+RecA was 10 ng/μL, then 10 μL of 20 ng/μL ssDNA+RecA solution was mixed with 10 μL of 24% (w/v) PVP in HEPES-CZB. A single spermatozoon moving slowly in the final 12% PVP solution was drawn, tail first, into the injection pipette in such a way that its neck (the junction between the head and tail) was at the opening of the pipette. The head was separated from the tail by applying a few piezo-pulses to the neck region. The tailless spermatozoon was then injected into an oocyte as described by Kimura and Yanagimachi. ICSI-oocytes were incubated in CZB medium at 37° C. under 5% CO₂ in air.

Embryo Culture and Embryo Transfer.

Oocytes with two well-developed pronuclei and a distinct second polar body 5 h after ICSI were recorded as being activated and were cultured in CZB medium until they reached the 2 cell stage (20-24 h after ICSI). They were then transferred into the oviducts of surrogate CD-1 females which were mated with vasectomized males of the same strain on the day before embryo transfer. Oocytes which were not to be transferred into surrogates were cultured in CZB medium until they reached the morula/blastocyst stage, and were observed for EGFP expression under fluorescent microscopy.

The data from a series of such experiments is tabulated in Table 1.

TABLE 1
Comparison of ICSI mediated transgenesis with Fresh and Freeze-thawed spermatozoa,
ssDNA and dsDNA, in the presence and absence of RecA recombinase.

| | | RecA/ | No. | | No. | | | | No. | | |
|---|---|---|---|---|---|---|---|---|---|---|---|
| Type of Sperm | | DNA | Oocytes | Survived | Fluorescing | % Green | Number | No. Live | Green | % Transgenic | % of Oocytes |
| Used | DNA | Ratio | Injected | Activation | at 4 Cell | Embryo | Transferred | Births | Pups | Pups | Injected |
| Fresh | ss | 0 | 139 | 86 | 21 | 25 | 84 | 42 | 0 | 0 | 0 |
| Freeze-thawed | ss | 0 | 67 | 32 | 6 | 18.75 | 29 | 13 | 0 | 0 | 0 |
| Fresh | ds | 0 | 61 | 43 | 6 | 13.9 | 39 | 8 | 0 | 0 | 0 |
| Freeze-thawed | ds | 0 | 59 | 41 | 10 | 24.4 | 24 | 0 | 0 | 0 | 0 |
| Fresh | ss | 80 to 1 | 190 | 158 | 53 | 33.5 | 117 | 38 | 7 | 18.4 | 3.7 |
| Freeze-thawed | ss | 80 to 1 | 47 | 32 | 14 | 43.8 | 23 | 5 | 1 | 20 | 2.1 |
| Fresh | ss | 40 to 1 | 76 | 66 | 14 | 21.2 | 44 | 20 | 5 | 25 | 6.6 |

These data demonstrate that the injection of either ds or ss DNA alone with live-fresh-sperm does lead to live born pups, but not transgenic ones. Therefore, the data indicates that the DNA used at the present concentrations of 10 ng/μL is not toxic for the production of live-born pups. Freeze-thawed sperm and ssDNA injection also allows the development of non transgenic pups to live births, comparable to live sperm births, whereas freeze-thawed sperm with dsDNA, at this reduced number of oocytes injected, does not. Note that in this case, dsDNA was injected into the oocytes, as well, as a control. Therefore, these results using freeze-thawed spermatozoa with dsDNA does not compare directly to conventional ICSI-Tr as previously reported (Perry 1999). Freeze-thawed sperm with ssDNA and RecA not only supports the development of live born pups, but also results in transgenic animals. RecA complexed to ssDNA and injected with fresh sperm lead to live born transgenic pups. All transgenic offspring produced under these conditions were fertile and passed the transgene to their offspring. A single chimeric animal produced with one of the 80:1 RecA:ssDNA conditions, using live sperm as a vector, was also able to produce fully transgenic offsprings, indicating that the germ line cells of this animal were transgenic for EGFP. These results confirm that fresh spermatozoa are more favorable than freeze-thawed spermatozoa for development to proceed, with a success rate ranging from 4% to 7% of total oocytes injected, probably as a result of a lower number of chromosomal aberrations. Secondly, they confirm that RecA is active in mouse transgenesis, because no transgenic pups were born with ssDNA alone, but 7 were obtained when RecA was complexed to ssDNA before injection at 80:1 ratio, and 5 live born pups were obtained in the 40:1 ratio. Third, the data suggest that our new method of RecA-ICSI-Tr is more efficient than conventional ICSI-Tr, which results in 2% of the total oocytes injected producing transgenic pups (Perry 1999).

### Example 3

### Homologous Recombination Using RecA-Mediated ICSI

The use of the bacterial recombinase protein RecA and ssDNA during ICSI transgenesis has permitted the use of a considerable lower number of MII oocytes for the production of transgenic mice, when compared to traditional ICSI. This Example tests whether early mouse embryos are competent for recombination and whether RecA can induce this recombination.

In brief, the buffer system for the incubation contains 25 mM Tris-HCl pH8.0, 150 mM KCl, 2 mM MgCl₂ (TKM buffer). The 50% glycerol is removed from the stock RecA supply by centrifugation through an Amersham Biosciences microspin column (G-25) as described above. The column is washed two times with TKM buffer and the amount of RecA to be used is mixed with an equal volume of 2×TKM. The mixture is placed in the middle of the column bed and centrifuged for 2 minutes at 735 g. The flow-through contains RecA at 50% of its original concentration and is adjusted accordingly for the 80:1 ratio incubation.

The ssDNA is prepared by taking an aliquot of the stock, usually a 2× concentration of the needed amount. It is mixed with TKM to bring it to the desired volume. It is then incubated at 95° C. (e.g., in a heat block) for 5 minutes, quenched on ice, the RecA solution added to it in the desired volume for the 80:1 ratio justification and incubated on ice for 60 minutes.

Spermatozoa were collected by the swim-up method from B6D2F1 mice as described previously. They were mixed with the RecA:ssDNA solution in a 50:50 ratio. ICSI-Tr was performed by injecting the sperm-RecA-ssDNA complex into oocytes obtained from superovulated CD-1 female mice. Although the Examples above illustrate the invention using an exogenous reporter gene, the present inventors expect that the methodology can be easily applied by one of ordinary skill in the art in light of the present teachings to obtain homologous recombination with transgene sequences that are complementary to the host genome, even producing transgenic animal with single point mutations (or correcting single point mutations).

### Example 4

### Characterization of Integration of ssDNA in RecA Mediated ICSI Transgenesis

This Example relates to the characterization of the sites of integration for ss-transgenes incorporated into the host genome via RecA mediated transgenesis during ICSI.

Tail clippings of transgenic animals produced with RecA mediated ICSI transgenesis are collected to analyze the number of integration sites in the genomic DNA extracted form this tissue. Analysis is carried out by Southern blotting or inverse PCR to identify regions of insertion into the mouse genome. Aliquots of genomic DNA are digested with blunt end cutting restriction enzymes which are not active in the transgene region, in this particular example with tyrosinase. The fragments are ligated in order to circularize the DNA and inverse PCR reactions are performed with primers mapping to the tyrosinase gene region. PCR products are optionally sequenced with the primers used for PCR.

A total of 8 animals were analyzed by Southern blotting. The transgene copy number varied from 2 to 20 copies in different animals. In all cases analyzed the copies appear to be passed on to the progeny and segregate as a single locus implying a tandem transgene array.

Confirmation of the integration sites affirms the occurrence of homologous recombination in a site-specific manner. The mutation of the tyrosinase allele as illustrated in this particular system will lead to the creation of a Spel restriction site that can be detected by PCR or Southern blot analysis.

In summary, the RecA-ICSI methodology can be applied in the production of knock-out mice in biomedicine as well as lead to an understanding of the steps involved in homologous recombination.

All publications and patents mentioned herein are hereby incorporated herein by reference in their entirety as if referred to individually.

What is claimed is:

1. A method for obtaining a transgenic cell, comprising the steps of:
(a) incubating a disease relevant exogenous nucleic acid with a recombinase;
(b) incubating the nucleic acid recombinase complex with a spermatozoon or sperm head;
(c) introducing a said spermatozoon or a sperm head into unfertilized oocyte of the same species to form a transgenic fertilized oocyte; and
(d) allowing the transgenic fertilized oocyte to develop into transgenic cells suitable for use in treating human diseases.

2. The method of claim 1, wherein said recombinase is E. coli RecA.

3. The method of claim 1, wherein said spermatozoon is live.

4. The method claim 1, wherein step (b) and step (c) are carried out simultaneously.

5. The method of claim 1, comprising preparing the sperm head from a fresh spermatozoon prior to step (c).

6. The method of claim 1, wherein the spermatozoon is introduced by in vitro fertilization.

7. The method of claim 1, wherein the unfertilized oocyte is a metaphase II oocyte.

8. The method of claim 1, wherein the exogenous nucleic acid comprises at least one human gene sequence.

9. The method of claim 1, wherein the oocyte and the spermatozoon or sperm head are from a mammal.

10. A transgenic cell produced according to the method of claim 1.

11. A method for obtaining a transgenic embryo, comprising the steps of:
(a) incubating an exogenous nucleic acid with a recombinase;
(b) incubating the nucleic acid recombinase complex with a spermatozoon or sperm head;
(c) introducing a said spermatozoon or a sperm head derived therefrom into unfertilized oocyte of the same species to form a transgenic fertilized oocyte; and
(d) allowing the transgenic fertilized oocyte to develop into transgenic embryo.

12. The method of claim 11, comprising transplanting the transgenic embryo into a surrogate mother to produce a transgenic animal.

13. The method of claim 11, wherein said transgenic animal is a model for human disease.

14. The method of claim 13, wherein said human disease is selected from the group consisting of cardiovascular diseases, neurological diseases, reproductive disorders, cancers, eye diseases, endocrine disorders, pulmonary diseases, metabolic disorders, autoimmune disorders, and aging.

15. The method of claim 14, wherein said transgenic animal is a model to demonstrate the safety and efficacy of one or more of the treatments selected from the group comprising drug therapy, gene therapy, stem cell therapy, and somatic cell therapy.

16. The method of claim 15, wherein said transgenic animal is a model for disease diagnosis.

17. A transgenic animal produced according to the method of claim 11.

18. The method of claim 11, further comprising the step of allowing the transgenic embryo to develop into a live offspring

19. A method of producing transgenic primate cells, comprising the steps of:
(a) incubating an exogenous nucleic acid with a recombinase;
(b) incubating the nucleic acid recombinase complex with a spermatozoon or sperm head
(c) introducing a said spermatozoon or a sperm head into unfertilized oocyte of the same species to form a transgenic fertilized oocyte; and
(d) allowing the transgenic fertilized oocyte to develop into transgenic cells suitable for use in treating human diseases.

20. A transgenic cell produced according to the method of claim 19.
